(19) United States
(12) Patent Application Publication
(10) Pub. No.: US 2023/0103319 A1
(43) Pub. Date: Apr. 6, 2023

(54) METHODS AND SYSTEMS FOR RISK ASSESSMENT OF ISCHEMIC CEREBROVASCULAR EVENTS

(71) Applicant: See-Mode Technologies Pte Ltd, Singapore (SG)

(72) Inventors: Sadaf MONAJEMI, Singapore (SG); Milad MOHAMMADZADEH, Singapore (SG)

(21) Appl. No.: 17/795,696

(22) PCT Filed: Feb. 1, 2021
(86) PCT No.: PCT/SG2021/050047

§ 371 Date: Jul. 27, 2022

(30) Foreign Application Priority Data
Jan. 31, 2020 (SG) 10202000904R

(51) Int. Cl. A61B 5/00; A61B 5/02

(52) U.S. Cl. CPC A61B 5/0042; A61B 5/02

(57) ABSTRACT

Systems, methods and a non-transitory computer readable medium for ischemic cerebrovascular event risk assessment from a vascular image of a patient are described. The vascular image of the patient includes at least a vessel and atherosclerotic plaque in the vessel and the method includes extracting hemodynamic information of the patient from the vascular image and extracting plaque information of the patient from the vascular image. The method also includes outputting a result indicating a risk of the ischemic cerebrovascular event using an artificial intelligence model based on one or both of the hemodynamic information and the plaque information. The system includes a medical imaging device, at least one processor and at least one memory. The memory(s) includes computer program code and the processor(s) and the computer code are configured to cause the system to extract hemodynamic information of the patient from the vascular image of the patient, extract plaque information of the patient from the vascular image of the patient and output a result indicating a risk of the ischemic cerebrovascular event using an artificial intelligence model based on at least the hemodynamic information or the plaque information.

### TECHNICAL FIELD

The present invention relates broadly, but not exclusively, to methods and systems for assessment of risk of an ischemic cerebrovascular event, such as a stroke, based on medical imaging data.

### BACKGROUND

Stroke is a leading cause of death globally, with over fifty per cent chance of death or disability within one year after occurrence. More than eighty-five per cent of strokes are ischemic, meaning that they are due to a blockage in blood flow to the brain. Ischemic strokes mainly occur due to atherosclerotic plaque rupturing in cerebrovascular arteries.

Two important parameters affect the risk of plaque rupture leading to ischemic stroke: first, the composition of the plaque, and second, the hemodynamic stress exerted on the plaque by blood flow through the cerebrovascular artery in which the plaque is located. Existing medical imaging modalities do not provide such information. Ultrasound (US), computerized tomography (CT), and magnetic resonance (MR) imaging are mainly focused on visualising the vascular lumen and grading the percentage of the narrowing in arteries. Thus, these medical imaging modalities provide anatomical information, but very limited functional information.

The functional information provided with common clinical tools is limited to blood flow velocity measurements (e.g. Doppler ultrasound). Additionally, the plaque composition is commonly determined through a subjective guess based on the appearance of plaque in ultrasound images. Such limited and subjective information is not enough for an accurate stroke risk assessment and could be a likely cause of the current twenty-five per cent stroke recurrence rate.

Accordingly, what is needed is a system and method for improving risk assessment for stroke or other ischemic cerebrovascular events (for example, transient ischemic attack (TIA)) that seek to address one or more of the above-mentioned problems. Furthermore, other desirable features and characteristics will become apparent from the subsequent detailed description and the appended claims, taken in conjunction with the accompanying drawings and this background of the disclosure.

### SUMMARY

According to a first aspect, a method for obtaining hemodynamic information of a patient is provided. The method includes providing a vascular medical image, the vascular medical image comprising at least a vessel. The method also includes segmenting the vessel in the vascular medical image and simulating blood flow based on a computational mesh generated on the segmented vessel.

According to another aspect, a method for obtaining vascular plaque information of a patient is provided. The method includes providing a vascular image, the vascular image comprising at least an atherosclerotic plaque in a vessel and segmenting the atherosclerotic plaque in the vascular medical image. The method also includes determining a plaque burden of the atherosclerotic plaque in the vessel based on data from the segmented atherosclerotic plaque and determining a material composition of the atherosclerotic plaque in the vessel based on data from the segmented atherosclerotic plaque.

According to a further aspect, there is provided a method for ischemic cerebrovascular event risk assessment from a vascular image of a patient. The vascular image includes at least a vessel and atherosclerotic plaque in the vessel and the method includes extracting hemodynamic information of the patient from the vascular image and extracting plaque information of the patient from the vascular image. The method also includes outputting a result indicating a risk of the ischemic cardiovascular event using an artificial intelligence model based on one or both of the hemodynamic information and the plaque information.

According to a yet another aspect, there is provided a system for ischemic cerebrovascular event risk assessment. The system includes a medical imaging device, at least one processor and at least one memory. The medical imaging device is configured to provide a vascular image of at least a vessel and atherosclerotic plaque in the vessel of a patient. The processor(s) is in communication with the medical imagining device. The memory(s) includes computer program code. The processor(s) and the computer code are configured to cause the system to extract hemodynamic information of the patient from the vascular image of the patient, extract plaque information of the patient from the vascular image of the patient and output a result indicating a risk of the ischemic cerebrovascular event using an artificial intelligence model based on at least the hemodynamic information or the plaque information.

According to yet a further aspect, there is provided a system for ischemic cerebrovascular event risk assessment from a vascular image of a patient, the vascular image including at least a vessel and atherosclerotic plaque in the vessel. The system includes a hemodynamic module, a plaque determination module and an artificial intelligence module. The hemodynamic module extracts hemodynamic information of the patient from the vascular image of the patient. The plaque determination module extracts plaque information of the patient from the vascular image of the patient. The artificial intelligence module outputs a result indicating a risk of the ischemic cerebrovascular event using an artificial intelligence model based on at least the hemodynamic information from the hemodynamic module or the plaque information from the plaque determination module.

According to a final aspect, there is provided a non-transitory computer readable medium having stored thereon an application which when executed by a computer causes the computer to perform ischemic cerebrovascular event risk assessment from a vascular image of a patient. The vascular image includes at least a vessel and atherosclerotic plaque in the vessel. The application when executed by the computer causes the computer to perform the steps of extracting hemodynamic information of the patient from the vascular image, extracting plaque information of the patient from the vascular image, and outputting a result indicating a risk of the ischemic cerebrovascular event using an artificial intelligence model based on at least the hemodynamic information or the plaque information.

### BRIEF DESCRIPTION OF DRAWINGS

Embodiments of the disclosure will be better understood and readily apparent to one of ordinary skill in the art from the following written description, by way of example only, and in conjunction with the drawings, in which:

FIG. 1 depicts a flowchart illustrating a method for improved risk assessment for stroke or other ischemic cerebrovascular events in accordance with present embodiments.

FIG. 2 depicts a block diagram of a module for computational hemodynamic simulation for blood flow in a system for improved risk assessment for stroke or other ischemic cerebrovascular events in accordance with the present embodiments.

FIG. 3, comprising FIGS. 3A to 3D, depicts illustrations of the steps for computational hemodynamic simulation for blood flow in the module of FIG. 2 in accordance with the present embodiments.

FIG. 4 depicts a block diagram of a module for plaque composition determination in the system for improved risk assessment for stroke or other ischemic cerebrovascular events in accordance with the present embodiments.

FIG. 5, comprising FIGS. 5A to 5D, depicts illustrations of the steps for plaque composition determination in the module of FIG. 4 in accordance with the present embodiments.

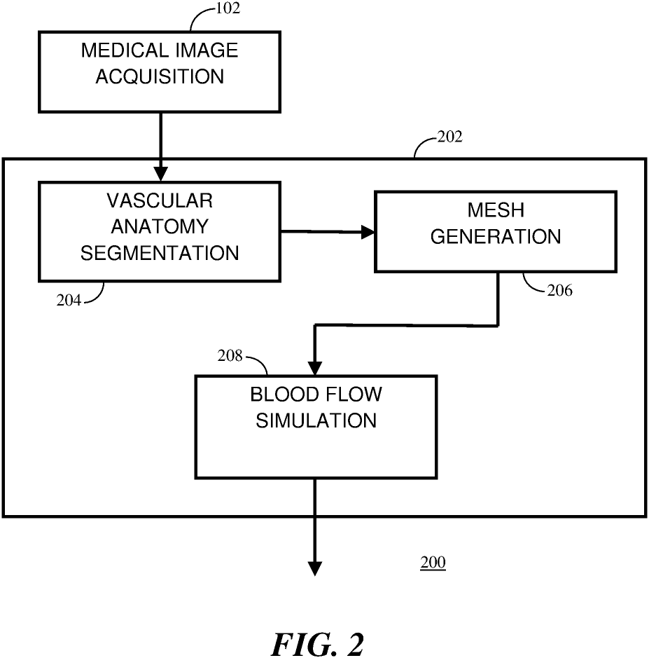

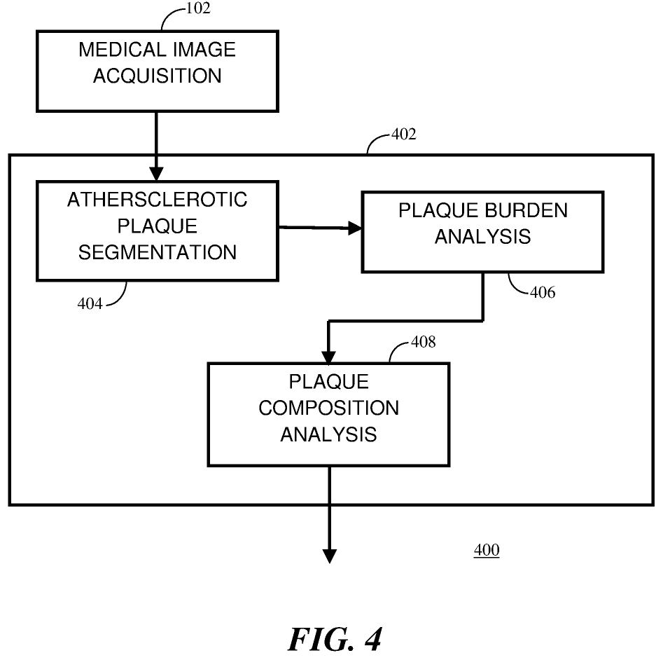

And FIG. 6 depicts an illustration of a module for stroke risk assessment using the combined information of the modules of FIGS. 2 and 4 in accordance with embodiments of the disclosure.

Skilled artisans will appreciate that elements in the figures are illustrated for simplicity and clarity and have not necessarily been depicted to scale. For example, the dimensions of some of the elements in the illustrations, block diagrams or flowcharts may be exaggerated in respect to other elements to help to improve understanding of the present embodiments.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description. It is the intent of the present embodiments to present a system and method to determine hemodynamic parameters and plaque composition from patient-specific medical data to help neurologists and other medical professional determine an optimum treatment plan for stroke patients or asymptomatic patients with a high risk of having a stroke. The systems and methods in accordance with present embodiments include acquisition of medical images from the patient’s cerebral vasculature, image analysis for three-dimensional reconstruction of the vasculature, blood flow analysis using computational fluid dynamics, detecting atherosclerotic plaques and their composition by analysing two-dimensional or three-dimensional medical images, and combining the plaque composition with the blood flow information for stroke risk assessment. The method uses non-invasive, post-processing computing techniques to determine a patient’s stroke risk from hemodynamic and plaque composition information. The hemodynamics information may include velocity, pressure, flow rate, shear stress, and any derivatives related to cerebrovascular arteries. The plaque composition may include information regarding the extent of calcification or intraplaque hemorrhage (IPH) within the same cerebrovascular arteries.

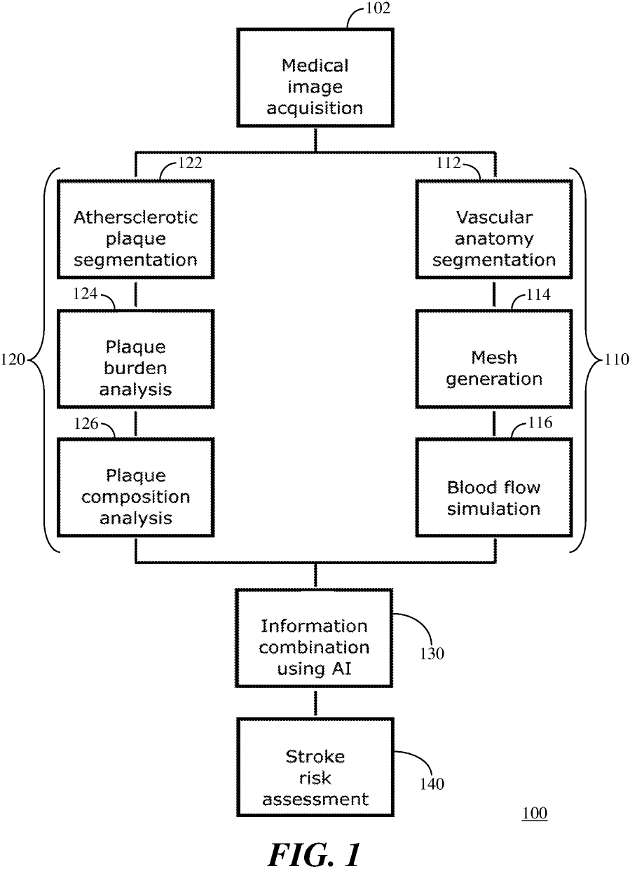

Referring to FIG. 1, a flow diagram 100 depicts a method for improving risk assessment for stroke or other ischemic cerebrovascular events in accordance with the present embodiments. Initially, patient-specific data such as medical images can be used for obtaining hemodynamic and plaque information to determine the stroke risk of the patient in accordance with the present embodiments. The medical images of a patient’s cerebral vasculature are acquired 102. These medical images may include ultrasound images, computed tomography (CT) images, or magnetic resonance imaging (MRI) images. Those skilled in the art will realize that the medical images acquired 102 may include images obtained from other medical imaging technologies so long as these medical images obtained from the patient are capable of computer analysis to segment cerebrovascular anatomy.

A three-dimensional reconstruction of cerebrovascular arteries is extracted from these images and used as the geometry for a computational fluid dynamics (CFD) simulation 110 including the steps of vascular anatomy segmentation 112, computational vascular mesh generation 114 based on the segmented vasculature, and vascular blood flow simulation 116 using the generated vascular mesh to provide detailed patient-specific hemodynamics information. This hemodynamic information may include pressure drop, fractional flow reserve (FFR), shear stress ratio (SSR), wall shear stress (WSS), or velocity ratio across the stenosis. Additionally, in accordance with the present embodiments, atherosclerotic plaque in cerebrovascular arteries is determined 120 from the medical images 102. Using image processing, the composition of the detected plaques is extracted from the medical images of the patient obtained at step 102 by segmenting 122 atherosclerotic plaque in the cerebrovascular arteries. The dimensions of the plaque or its volume may be used for determining the plaque burden in the segmented cerebrovascular arteries 124. The material composition of the plaque can then be determined by performing a plaque composition analysis 126 using image processing and machine learning.

The hemodynamic information from the blood flow simulation 116 is then combined 130 with the analysis 126 of the atherosclerotic plaque composition to determine plaque vulnerability using an artificial intelligence model to stratify, assess and/or classify 140 the stroke risk of the patient.

The method described in the flow diagram 100 provides a more accurate risk assessment for stroke occurrence or recurrence compared to using only anatomical information. The medical image 102 can be data obtained from existing medical data of patients without the need for performing new tests. The non-invasive nature of the method described in the flow diagram 100 advantageously limits the risks associated with invasive vascular measurements for determining hemodynamic information like the use of catheter-based pressure probes for measuring FFR.

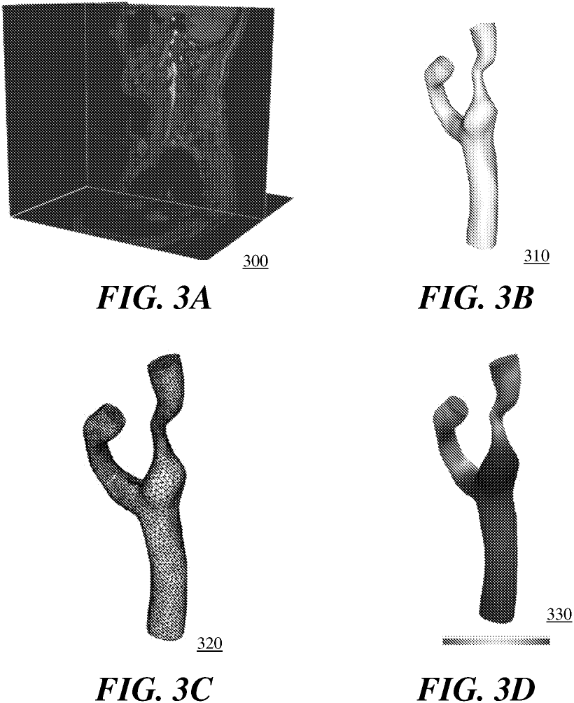

Referring to FIG. 2, a block diagram 200 of a module 202 for hemodynamic analysis in a system for improved risk assessment for stroke or other ischemic cerebrovascular events in accordance with the present embodiments is depicted. Referring to FIGS. 3A to 3D illustrations 300, 310, 320, 330 depict the steps for hemodynamic analysis in the module 202 in accordance with the present embodiments. The image information 102 used by the module 202 may be cerebrovascular images such as volumetric angiography images 300 (FIG. 3A) obtained for example through computerized tomography (CT) or magnetic resonance (MR) imaging. The lumen can be extracted from the cerebrovascular images to segment and reconstruct a vessel of interest as a three-dimensional (3D) object 310 (FIG. 3B) by a vascular segmentation process 204, which is then used for generation of a volumetric mesh 320 (FIG. 3C) by a mesh generation process 206. Blood flow in the segmented vasculature can be simulated using a patient-specific computational fluid dynamics model 208, the results of which may be shown with colormaps 330 (FIG. 3D), or summary tables of important information such as FFR, SSR, WSS, velocity ratio, or pressure drop across a stenosis. Hemodynamic information alone may be used for stroke risk assessment of the patient in accordance with the present embodiments, or the hemodynamic information may be combined with other risk factors such as plaque composition as discussed hereinafter.

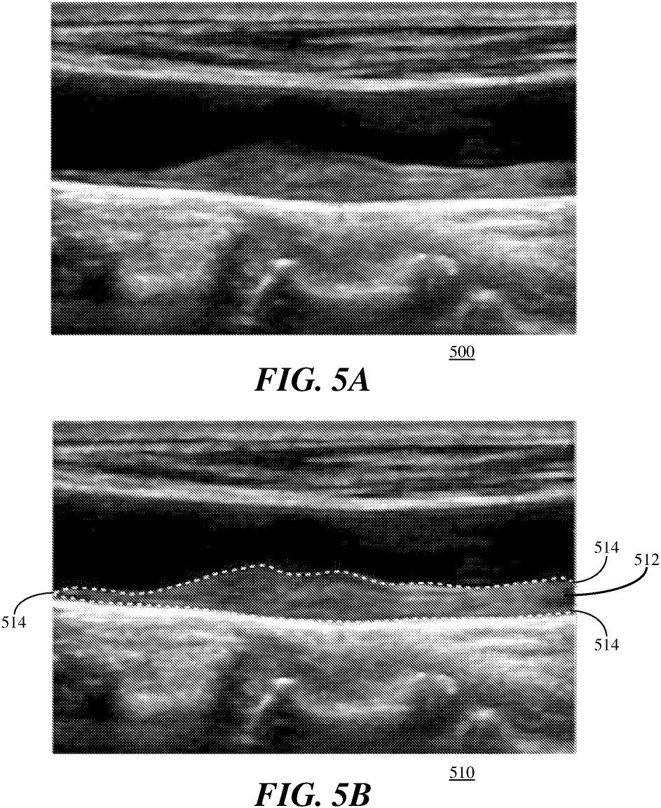

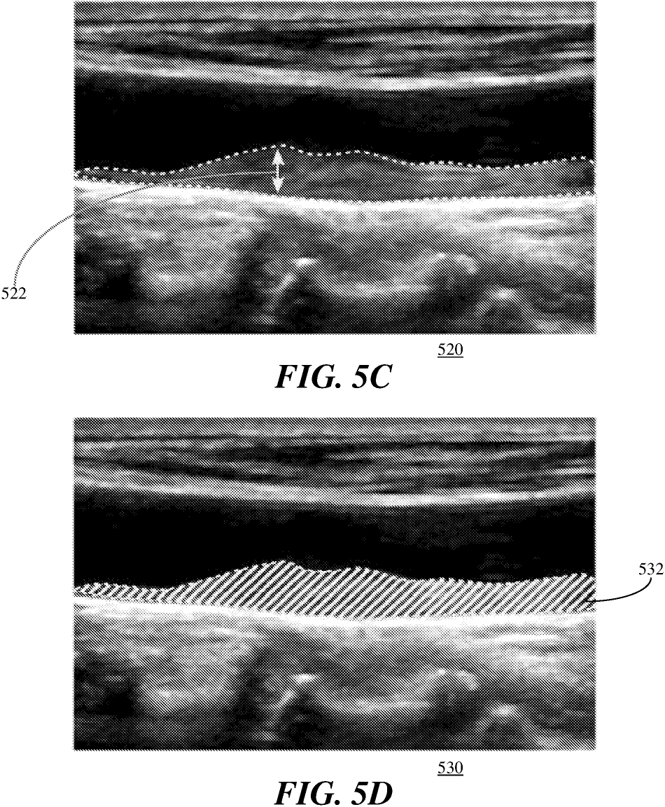

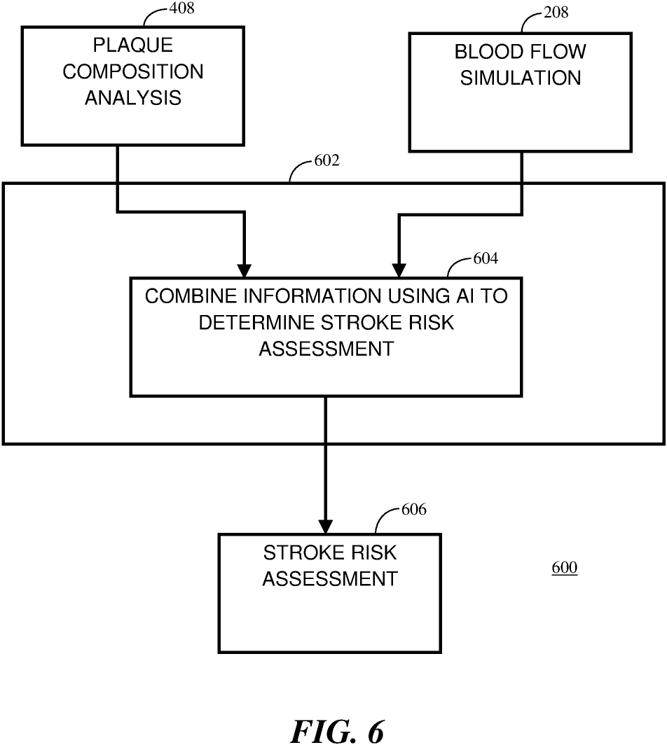

In addition to volumetric modalities like CT and MR, two-dimensional images like longitudinal and transverse B-Mode ultrasound may be used for stroke risk assessment. Referring to FIG. 4, a block diagram 400 of a module 402 for plaque composition determination in a system for improved risk assessment for stroke or other ischemic cerebrovascular events in accordance with the present embodiments is depicted. Referring to FIGS. 5A to 5D illustrations 500, 510, 520, 530 depict the steps for plaque composition determination in the module 402 in accordance with the present embodiments. FIG. 5A shows analysis of a longitudinal B-Mode ultrasound image 500 for segmentation of atherosclerotic plaque by an image segmentation process 404 to obtain the image 510 (FIG. 5B) segmenting the atherosclerotic plaque 512 within the dashed line 514. The plaque burden, including the atherosclerotic plaque axial, lateral, or volumetric dimensions (as shown in the image 520 (FIG. 5C) where the axial dimension is indicated by an arrow 522), can be calculated by a plaque burden analysis 406. Finally, the plaque composition 532 in the image 530 (FIG. 5D) may be obtained by image processing and machine learning techniques of a plaque composition analysis 408, using intensity and texture features such as plaque morphology and gray scale median in the segmented plaque area in comparison with an external database of labeled plaque information. This information may be used to determine the vulnerability of the atherosclerotic plaque and its chance of rupture. The patient’s stroke risk may be determined by using the plaque information alone in accordance with the present embodiments.

Alternatively, FIG. 6 depicts an illustration 600 of a module 602 for stroke risk assessment using the combined information of the modules 202 (FIG. 2) and 402 (FIG. 4) in accordance with the present embodiments. The results of the plaque composition analysis 408 and the results of the blood flow simulation 208 are inputted to the module 602 where the an AI model 604 combines the plaque information from the plaque composition analysis 408 with the patient-specific hemodynamic parameters obtained from the blood flow simulation 408 to assess stroke risk of the patient. The stroke risk assessment 606 is then outputted from the system.

Thus, it can be seen that a system and method for improved risk assessment for stroke or other ischemic cerebrovascular events such as TIAs has been provided which provides improved and robust systems and methods for more accurate risk assessment for stroke occurrence or recurrence compared to conventional systems which use only anatomical information. The method and system can be applied on existing medical data of patients without the need for receiving new tests. The non-invasive nature of the method limits the risks associated with invasive vascular measurements for determining hemodynamic information like the use of catheter-based pressure probes.

It will be appreciated by a person skilled in the art that numerous variations and/or modifications may be made to the present disclosure as shown in the specific embodiments without departing from the spirit or scope of the disclosure as broadly described. The present embodiments are, therefore, to be considered in all respects to be illustrative and not restrictive.

1. A method to assess ischemic cerebrovascular event risk from a volumetric cerebrovascular image of a patient, wherein the volumetric cerebrovascular image comprises at least a cerebral vessel and atherosclerotic plaque in the cerebral vessel, the method comprising:
extracting cerebral vessel hemodynamic information of the patient from the cerebral vessel in the volumetric cerebrovascular image;
extracting cerebral vessel plaque information of the patient from the atherosclerotic plaque in the cerebral vessel in the volumetric cerebrovascular image; and
outputting a result indicating a risk of the ischemic cerebrovascular event using an artificial intelligence model based on both the hemodynamic information and the plaque information.

2. The method as claimed in claim 1, wherein the step of extracting cerebral vessel hemodynamic information of the patient comprises:
segmenting the cerebral vessel in the volumetric cerebrovascular image; and
simulating blood flow in the cerebral vessel based on a computational mesh generated on the segmented cerebral vessel.

3. The method as claimed in claim 2, wherein the step of simulating blood flow in the cerebral vessel based on a computational mesh comprises simulating blood flow in the cerebral vessel based on a three-dimensional volumetric mesh generated on the segmented cerebral vessel.

4. The method as claimed in claim 3, wherein the step of simulating blood flow in the cerebral vessel comprises simulating blood flow in the cerebral vessel using a patient-specific computational fluid dynamics model.

5. The method as claimed in claim 1, wherein the step of extracting cerebral vessel plaque information of the patient comprises:
segmenting the atherosclerotic plaque in the cerebral vessel in the volumetric cerebrovascular medical image;
determining a plaque burden of the atherosclerotic plaque in the cerebral vessel based on data from the segmented atherosclerotic plaque; and
determining a material composition of the atherosclerotic plaque in the cerebral vessel based on the segmented atherosclerotic plaque.

6. The method as claimed in claim 5, wherein determining the material composition of the atherosclerotic plaque in the cerebral vessel comprises determining the material composition of the atherosclerotic plaque in the vessel based on data of the segmented atherosclerotic plaque, the data of the segmented atherosclerotic plaque comprising one or more of dimensions, volume, morphology, texture, and intensity features of the atherosclerotic plaque.

7. The method as claimed in claim 6, wherein the steps of determining the plaque burden and determining the material composition comprise using image processing and machine learning methods to determine the plaque burden and the material composition.

8. The method as claimed in claim 1, wherein extracting the cerebral vessel hemodynamic information comprises extracting hemodynamic information selected from the group consisting of velocity, pressure, flow rate, and derivatives of velocity, pressure and flow rate including wall shear stress (WSS), pressure drop, fractional flow reserve (FFR), shear stress ratio (SSR), or velocity ratio across a stenosis.

9. The method as claimed in claim 1, wherein extracting the cerebral vessel plaque information of the patient comprises extracting plaque composition selected from the group consisting of extent of calcification and intraplaque haemorrhage (IPH).

10. The method as claimed in claim 9, wherein the volumetric cerebrovascular image comprises a computed tomography (CT) image or a magnetic resonance (MR) image.

11. An ischemic cerebrovascular event risk assessment system to assess ischemic cerebrovascular event risk, the system comprising:
a medical imaging device configured to provide a volumetric cerebrovascular image of a patient, wherein the volumetric cerebrovascular image comprises volumetric cerebrovascular information of at least a cerebral vessel and atherosclerotic plaque in the cerebral vessel;
at least one processor in communication with the medical imagining device; and
at least one memory including computer program code, the at least one memory and the computer program code configured to, with the at least one processor, cause the system at least to:
extract hemodynamic information of the patient from the volumetric cerebrovascular information;
extract plaque information of the patient from the volumetric cerebrovascular information; and
output a result indicating a risk of the ischemic cerebrovascular event using an artificial intelligence model based on at least the extracted hemodynamic information and the extracted plaque information.

12. The system as claimed in claim 11, wherein the medical imaging device comprises a computed tomography (CT) imaging device or a magnetic resonance (MR) imaging device.

13. An ischemic cerebrovascular event risk assessment system to assess ischemic cerebrovascular event risk from a volumetric cerebrovascular image of a patient, the volumetric cerebrovascular image comprising at least a cerebral vessel and atherosclerotic plaque in the cerebral vessel, the system comprising:
a hemodynamic module configured to extract hemodynamic information of the patient from the cerebral vessel in the volumetric cerebrovascular image of the patient;
a plaque determination module configured to extract plaque information of the patient from the atherosclerotic plaque in the cerebral vessel in the volumetric cerebrovascular image of the patient; and
an artificial intelligence module coupled to the hemodynamic module and the plaque determination module and configured to generate and output a result indicating a risk of the ischemic cerebrovascular event using an artificial intelligence model based on at least the hemodynamic information from the hemodynamic module and the plaque information from the plaque determination module.

14. The system as claimed in claim 13, wherein the hemodynamic module comprises:
a vascular anatomy segmentation module for segmenting the cerebral vessel in the volumetric cerebrovascular image;
a mesh generation module coupled to the vascular anatomy segmentation module for generating a three-dimensional volumetric mesh on the segmented cerebral vessel; and
a blood flow simulation module coupled to the mesh generation module for generating the hemodynamic information by simulating blood flow in the cerebral vessel based on the computational mesh using a patient-specific computational fluid dynamics model.

15. The system as claimed in claim 13, wherein the plaque determination module comprises:
an atherosclerotic plaque segmentation module for segmenting the atherosclerotic plaque in the cerebral vessel in the volumetric cerebrovascular image;
a plaque burden analysis module for using image processing and/or machine learning methods to determine a plaque burden of the atherosclerotic plaque in the cerebral vessel based on data from the segmented atherosclerotic plaque; and
a plaque burden analysis module for using image processing and/or machine learning methods to determine the plaque information in response to determining a material composition of the atherosclerotic plaque in the cerebral vessel based on the segmented atherosclerotic plaque, the material composition of the atherosclerotic plaque in the vessel determined in response to data of the segmented atherosclerotic plaque, wherein the data of the segmented atherosclerotic plaque comprises one or more of dimensions, volume, morphology, and grey scale median of the atherosclerotic plaque.

16. The system as claimed in claim 13, wherein the hemodynamic information comprises pressure drop, fractional flow reserve (FFR), shear stress ratio (SSR), wall shear stress (WSS), and/or velocity ratio across a stenosis.

17. The system as claimed in claim 13, wherein the plaque information comprises plaque composition, extent of calcification and/or intraplaque haemorrhage (IPH).

18. The system as claimed in claim 13, wherein the volumetric cerebrovascular image comprises a computed tomography (CT) image or a magnetic resonance (MR) image.

19. A non-transitory computer readable medium having stored thereon an application which when executed by a computer causes the computer to assess ischemic cerebrovascular event risk from a volumetric cerebrovascular image of a patient, wherein the volumetric cerebrovascular image comprises a computed tomography (CT) image or a magnetic resonance (MR) image and includes at least an image of a cerebral vessel and atherosclerotic plaque in the cerebral vessel, the application when executed by the computer causes the computer to perform the steps comprising:
extract hemodynamic information of the patient from the image of the cerebral vessel in the volumetric cerebrovascular image;
extract plaque information of the patient from the image of the atherosclerotic plaque in the cerebral vessel in the volumetric cerebrovascular image; and
output a result indicating a risk of the ischemic cerebrovascular event using an artificial intelligence model based on at least the hemodynamic information and the plaque information.
